(19) United States
(12) Patent Application Publication
(10) Pub. No.: US 2026/0294615 A1
(43) Pub. Date: Oct. 1, 2026

(54) Functionalized Implant-Delivery Funnels and Methods

(71) Applicant: Silimed Indústria de Implantes Ltda., Rio de Janeiro (BR)

(72) Inventors: Gualtiero Victor Lopes Domingues, Rio de Janeiro (BR); Eduardo Pedro Milan, Rio de Janeiro (BR); Jeniffer Farias Dos Santos, Rio de Janeiro (BR)

(21) Appl. No.: 19/478,255

(22) PCT Filed: Apr. 25, 2024
(86) PCT No.: PCT/IB2024/000204

§ 371 Date: Oct. 23, 2025

Related U.S. Application Data
(60) Provisional application No. 63/461,861, filed on Apr. 25, 2023.

(51) Int. Cl. A61F 2/12; A61F 2/00

(52) U.S. Cl. CPC A61F 2/12; A61F 2/0095

(57) ABSTRACT

An implant-delivery funnel can include a funnel body, a funnel opening, and a funnel exit, the funnel opening and exit at opposite ends the funnel body. The funnel body can include an outer base layer and an inner coating layer anchored to a functionalized mating face of the outer base layer. The inner coating layer can be hydrophilic so as to form a lubricious coating with retained water. The funnel opening can be sized to receive an implant therethrough without any permanent deformation of the implant. The funnel exit can be adjustably sized to deliver the implant squeezed therethrough with a temporary reduction in size of the implant. A method of making an implant-delivery funnel can include functionalizing the outer base layer and applying the inner coating layer over a functionalized mating face of the outer base layer, thereby anchoring the inner coating layer to the outer base layer.

### PRIORITY

This application claims the benefit of priority to U.S. Provisional Patent Application No. 63/461,861, filed Apr. 25, 2023, which is incorporated by reference in its entirety into this application.

### BACKGROUND

The use of silicone implants such as breast implants emerged in the 1960s as an evolution to formerly used methods of augmentation. Injections of substances such as glycerin, bovine cartilage, silicone oil, and even snake venom were previously used at significant risk until Cronin and Gerow created the silicone implants we know today, which envelope silicone gel within a thin layer of rigid silicone. (Perry, D., et al. The history and development of breast implants. The Annals of The Royal College of Surgeons of England. 2020, 102(7), 478-482.)

Despite several advances in silicone implants, there are still problems to overcome, one being that the silicone implants are pre-filled, thereby requiring them to be inserted into surgical pockets created therefor by relatively wide incisions. Such wide incisions can result in large, aesthetically unpleasant scars. Indeed, for women of Asian descent, it is commonly required that small incisions be made due to their propensity to develop fibrous tissue and hyperpigmentation around the incisions. Some have even opted for surgeries with trans-umbilical or axillary incisions (Chen, S. H., et al. Lubricating the insertion funnel with autologous fat tissue for inserting breast implants. Plastic and Reconstructive Surgery-Global Open. 2018, 6(2), e1641.). Another of the problems with silicone implants to overcome is that of contamination. Since the silicone implants are manually inserted by hand, there is a notable risk of contamination by surgeons' hands, which risk of contamination increases with handling and exposure time. Further to the silicone implants being manually inserted by hand, there is also notable risk of capsular contracture, which is believed to be caused by introducing skin flora into the surgical pockets during delivery of the silicone implants thereto.

Implant-delivery funnels for delivering the silicone implants to their corresponding surgical pockets require relatively smaller incisions, reduce handling and exposure time of the silicone implants, and reduce opportunities for contamination of the surgical pockets with skin flora by way of conduits to the surgical pockets created by the implant-delivery funnels. Notwithstanding the foregoing, the risks of contamination and capsular contraction remain. Further, existing implant-delivery funnels limit use or at least usefulness of certain silicone implants such as polyurethane-coated silicone implants, which implants are configured to reduce malposition in their surgical pockets or beyond by tissue adherence or ingrowth into the polyurethane thereof. Indeed, lubricants used within the existing implant-delivery funnels can bind to or fill surface pores of the polyurethane-coated silicone implants, thereby limiting opportunities for the tissue adherence or ingrowth into the polyurethane, which increases opportunities for malposition.

Disclosed herein are functionalized implant-delivery funnels and methods that address the foregoing.

### SUMMARY

Disclosed herein is an implant-delivery funnel including, in some embodiments, a supple, layered funnel body, a funnel opening at a proximal end of the funnel body, and a funnel exit at a distal end of the funnel body. The funnel body includes an outer base layer and an inner coating layer anchored to the outer base layer by dipole-dipole forces. The inner coating layer is hydrophilic such that the inner coating layer forms a lubricious coating with retained water. The funnel opening is sized to receive an implant therethrough without any permanent deformation of the implant. The funnel exit is adjustably sized to deliver the implant squeezed therethrough with a temporary reduction in size of the implant.

In some embodiments, the outer base layer includes one or more layers of polyvinyl chloride.

In some embodiments, the outer base layer includes at least one layer of polyethylene and at least one layer of polyester.

In some embodiments, the outer base layer is free of any phthalate plasticizers.

In some embodiments, the dipole-dipole forces include hydrogen bonding.

In some embodiments, a mating face of the outer base layer is functionalized with amino functional groups. The inner coating layer is anchored to the outer base layer by the hydrogen bonding.

In some embodiments, a mating face of the outer base layer is functionalized with hydroxyl functional groups. The inner coating layer is anchored to the outer base layer by the hydrogen bonding.

In some embodiments, a mating face of the outer base layer is functionalized with amino and hydroxyl functional groups. The inner coating layer is anchored to the outer base layer by the hydrogen bonding.

In some embodiments, the inner coating layer is hydrated with water or an aqueous mixture, thereby forming the lubricious coating with the retained water.

In some embodiments, the inner coating layer includes one or more layers of a cross-linked biopolymer forming a polymer network configured to swell with the retained water.

In some embodiments, the inner coating layer is further antimicrobial. The polymer network is configured to swell with the retained water and any antimicrobial dissolved or suspended in the aqueous mixture.

In some embodiments, the funnel body further includes an innermost antimicrobial layer.

In some embodiments, the funnel body further includes an outermost antimicrobial layer.

In some embodiments, the funnel body includes a longitudinal welded seam.

In some embodiments, the funnel body includes a transverse welded seam.

In some embodiments, the implant-delivery funnel is configured to deliver breast implants of various volumes and shapes.

In some embodiments, a distal portion of the funnel body includes indicia configured for cutting the distal portion of the funnel body to size such that the funnel exit is sized to deliver the implant squeezed therethrough with the temporary reduction in size of the implant.

Also disclosed herein is a method of making an implant-delivery funnel. The method includes, in some embodiments, a base layer-forming operation, a functionalizing operation, a coating layer-applying operation, and a welding operation. The base layer-forming operation includes forming an outer base layer of a funnel body. The functionalizing operation includes functionalizing a mating face of the outer base layer. The coating layer-applying operation includes applying an inner coating layer over a functionalized mating face of the outer base layer, thereby anchoring the inner coating layer to the outer base layer by dipole-dipole forces including hydrogen bonding. The welding operation includes welding together the funnel body to form a funnel opening at a proximal end of the funnel body and a funnel exit at a distal end of the funnel body. The funnel opening is sized to receive an implant therethrough without any permanent deformation of the implant. The funnel exit is adjustably sized to deliver the implant squeezed therethrough with a temporary reduction in size of the implant.

In some embodiments, the welding together of the funnel body in the welding operation is before the functionalizing of the mating face of the outer layer in the functionalizing operation.

In some embodiments, the welding together of the funnel body in the welding operation is after the functionalizing of the mating face of the outer layer in the functionalizing operation but before the applying of the inner coating layer over the functionalized mating face of the outer base layer in the coating layer-applying operation.

In some embodiments, the welding together of the funnel body in the welding operation is after both the functionalizing of the mating face of the outer layer in the functionalizing operation and the applying of the inner coating layer over the functionalized mating face of the outer base layer in the coating layer-applying operation.

In some embodiments, the welding together of the funnel body in the welding operation is by radiofrequency welding.

In some embodiments, the functionalized mating face of the outer base layer is functionalized with amino functional groups via plasma functionalization in an atmosphere of nitrogen. The inner coating layer is anchored to the outer base layer by the hydrogen bonding.

In some embodiments, the functionalized mating face of the outer base layer is functionalized with amino and hydroxyl functional groups via plasma functionalization in an atmosphere of natural or synthetic air. The inner coating layer is anchored to the outer base layer by the hydrogen bonding.

In some embodiments, the method further includes a hydrating operation. The hydrating operation includes hydrating the inner coating layer with water or an aqueous mixture to form a lubricious coating with retained water. The inner coating layer includes one or more layers of a cross-linked biopolymer forming a polymer network configured to swell with the retained water.

In some embodiments, the inner coating layer is further antimicrobial. The polymer network is configured to swell with the retained water and any antimicrobial dissolved or suspended in the aqueous mixture.

In some embodiments, the method further includes an antimicrobial-applying operation. The antimicrobial-applying operation includes applying an innermost antimicrobial layer.

In some embodiments, the method further includes an antimicrobial-applying operation. The antimicrobial-applying operation includes applying an outermost antimicrobial layer.

In some embodiments, the method further includes an indicia-creating operation. The indicia-creating operation includes creating indicia on an outer face of the outer base layer in a distal portion thereof. The indicia are configured for cutting the distal portion of the funnel body to size such that the funnel exit is sized to deliver the implant squeezed therethrough with the temporary reduction in size of the implant.

These and other features of the concepts provided herein will become more apparent to those of skill in the art in view of the accompanying drawings and following description, which describe particular embodiments of such concepts in greater detail.

### DRAWINGS

FIG. 1 illustrates an implant-delivery funnel in accordance with some embodiments.

FIG. 2 illustrates layers of a funnel body of the implant-delivery funnel in accordance with some embodiments.

FIG. 3 illustrates the layers of the funnel body in accordance with some other embodiments.

FIG. 4 illustrates the layers of the funnel body in accordance with yet some other embodiments.

FIG. 5 illustrates hydrogen bonding between the base layer and an inner coating layer of the funnel body in accordance with some embodiments.

FIG. 6 illustrates functionalization of a base layer of the funnel body in accordance with some embodiments.

FIG. 7 illustrates how the implant-delivery funnel functions to deliver an implant in accordance with some embodiments.

FIG. 8 illustrates implantation of an implant in a patient via delivery of the implant with the implant-delivery funnel in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

“Proximal” is used to indicate a portion, section, piece, element, or the like of a medical device intended to be near or relatively nearer to a clinician when the medical device is used on a patient. For example, a “proximal portion” or “proximal section” of the medical device includes a portion or section of the medical device intended to be near the clinician when the medical device is used on the patient. Likewise, a “proximal length” of the medical device includes a length of the medical device intended to be near the clinician when the medical device is used on the patient. A “proximal end” of the medical device is an end of the medical device intended to be near the clinician when the medical device is used on the patient. The proximal portion, the proximal section, or the proximal length of the medical device need not include the proximal end of the medical device. Indeed, the proximal portion, the proximal section, or the proximal length of the medical device can be short of the proximal end of the medical device. However, the proximal portion, the proximal section, or the proximal length of the medical device can include the proximal end of the medical device. Should context not suggest the proximal portion, the proximal section, or the proximal length of the medical device includes the proximal end of the medical device, or if it is deemed expedient in the following description, “proximal portion,” “proximal section,” or “proximal length” can be modified to indicate such a portion, section, or length includes an end portion, an end section, or an end length of the medical device for a “proximal end portion,” a “proximal end section,” or a “proximal end length” of the medical device, respectively.

“Distal” is used to indicate a portion, section, piece, element, or the like of a medical device intended to be near, relatively nearer, or even in a patient when the medical device is used on the patient. For example, a “distal portion” or “distal section” of the medical device includes a portion or section of the medical device intended to be near, relatively nearer, or even in the patient when the medical device is used on the patient. Likewise, a “distal length” of the medical device includes a length of the medical device intended to be near, relatively nearer, or even in the patient when the medical device is used on the patient. A “distal end” of the medical device is an end of the medical device intended to be near, relatively nearer, or even in the patient when the medical device is used on the patient. The distal portion, the distal section, or the distal length of the medical device need not include the distal end of the medical device. Indeed, the distal portion, the distal section, or the distal length of the medical device can be short of the distal end of the medical device. However, the distal portion, the distal section, or the distal length of the medical device can include the distal end of the medical device. Should context not suggest the distal portion, the distal section, or the distal length of the medical device includes the distal end of the medical device, or if it is deemed expedient in the following description, “distal portion,” “distal section,” or “distal length” can be modified to indicate such a portion, section, or length includes an end portion, an end section, or an end length of the medical device for a “distal end portion,” a “distal end section,” or a “distal end length” of the medical device, respectively.

Unless defined otherwise, all technical and scientific terms used herein have the same meaning as commonly understood by those of ordinary skill in the art.

As set forth above, while the existing implant-delivery funnels for delivering silicone implants to their corresponding surgical pockets reduce handling and exposure time of the silicone implants, as well as reduce opportunities for contamination of the surgical pockets with skin flora, the risks of contamination and capsular contraction remain. Further, the existing implant-delivery funnels limit use or at least usefulness of certain silicone implants such as polyurethane-coated silicone implants, which implants are configured to reduce malposition in their surgical pockets or beyond by tissue adherence or ingrowth into the polyurethane thereof. Indeed, the lubricants used within the existing implant-delivery funnels can bind to or fill the surface pores of the polyurethane-coated silicone implants, thereby limiting the opportunities for the tissue adherence or ingrowth into the polyurethane, which increases opportunities for malposition.

Disclosed herein are functionalized implant-delivery funnels and methods that address the foregoing. In an example, an implant-delivery funnel can include a funnel body, a funnel opening, and a funnel exit, wherein the funnel opening and the funnel exit are at opposite ends the funnel body. The funnel body can include an outer base layer and an inner coating layer anchored to a functionalized mating face of the outer base layer. The inner coating layer can be hydrophilic so as to form a lubricious coating with retained water. The funnel opening can be sized to receive an implant therethrough without any permanent deformation of the implant. The funnel exit can be adjustably sized to deliver the implant squeezed therethrough with a temporary reduction in size of the implant. In another example, a method of making an implant-delivery funnel can include functionalizing the outer base layer and applying the inner coating layer over the functionalized mating face of the outer base layer, thereby anchoring the inner coating layer to the outer base layer. These and other features of the implant-delivery funnels and methods provided herein will become more apparent to those of skill in the art in view of the accompanying drawings and following description, which describe particular embodiments in greater detail.

### Implant-Delivery Funnels

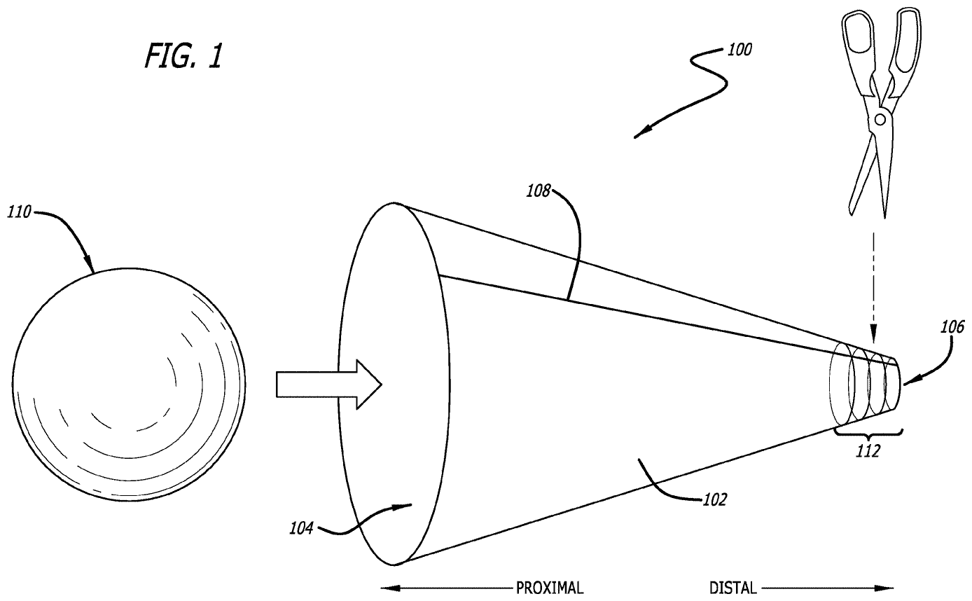

FIG. 1 illustrates an implant-delivery funnel 100 in accordance with some embodiments.

As shown, the implant-delivery funnel 100 can include a supple, layered funnel body 102, optionally, of sufficient translucency for implant visualization therethrough, a funnel opening 104 at a proximal end of the funnel body 102, and a funnel exit 106 at a distal end of the funnel body 102. Further, the implant-delivery funnel 100 can include a longitudinal seam 108 such as a welded seam, for example, a radiofrequency-welded seam. While not shown, the implant-delivery funnel 100 can also include a transverse seam such as a welded seam, for example, a radiofrequency-welded seam. Indeed, the implant-delivery funnel 100 can include a pair of transverse seams across proximal and distal portions of the funnel body 102 such as the proximal and distal end of the funnel body 102, thereby closing the proximal and distal portions of the funnel body 102, optionally, in a completely flat state of the implant-delivery funnel 100. Such transverse seams maintain sterility within the implant-delivery funnel 100 prior to using the implant-delivery funnel 100 as described in the method set forth below. Further, such transverse seams can assist in retaining the water or the aqueous mixture in the implant-delivery funnel 100 during the funnel-hydrating operation of the method set forth below.

The funnel opening 104 can be sized to receive an implant 110 therethrough without any permanent deformation of the implant 110. That is, the funnel opening 104 can be sized larger than the implant 110 such that the implant 110 need not be manipulated when dropped or inserted into the funnel opening 104. Such a funnel opening 104 facilitates dropping or inserting the implant 110 into the implant-delivery funnel 100 with minimal to no direct handling, thereby reducing any potential for contamination.

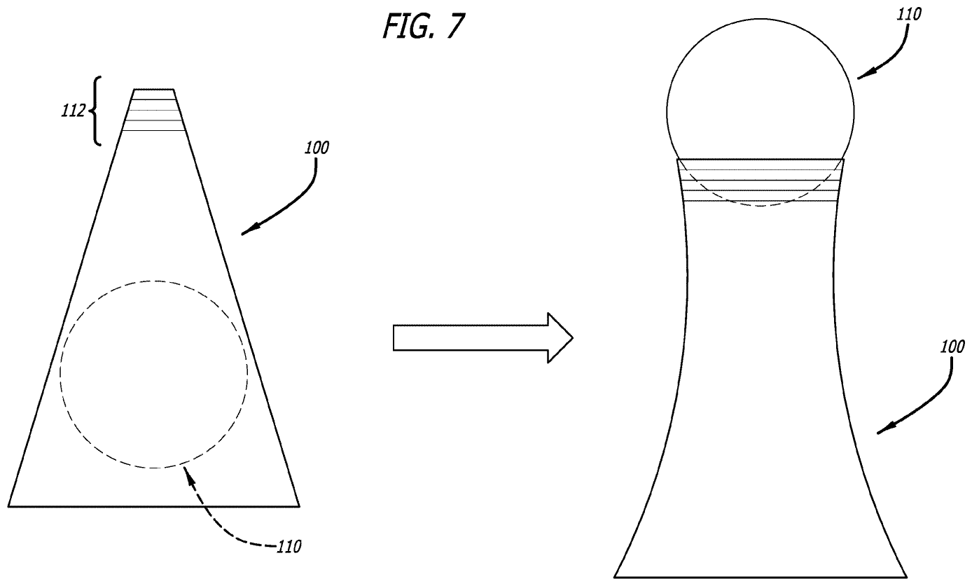

The funnel exit 106 can be adjustably sized to deliver the implant 110 squeezed therethrough with a temporary reduction in size of the implant 110. (See FIG. 7.) Indeed, the distal portion of the funnel body 102 can include indicia 112, for example, annular lines printed on an exposed, outer face 114 of the outer base layer 116, according to implant size, the indicia 112 configured for cutting the distal portion of the funnel body 102 to size such that the funnel exit 106 is appropriately sized to deliver the implant 110 squeezed therethrough with the temporary reduction in the size of the implant 110. As such, the implant-delivery funnel 100 can be configured to deliver implants such as breast implants of various volumes and shapes without permanently deforming, structurally weaking, or rupturing the implants, rupturing the funnel body 102, injuring patients, or the like.

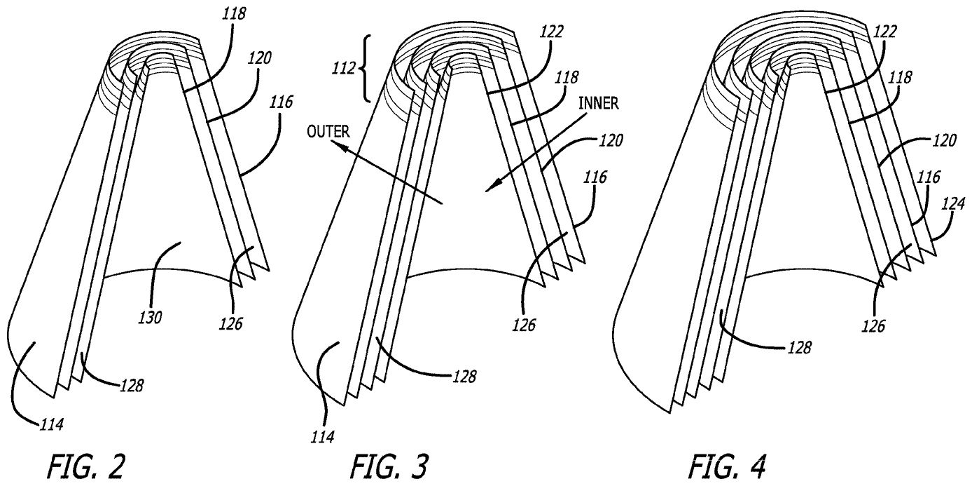

FIGS. 2-4 illustrate layers of the funnel body 102 of the implant-delivery funnel 100 in accordance with some embodiments.

As shown, the funnel body 102 can include an outer base layer 116 and an inner coating layer 118 anchored to the outer base layer 116 at an interface 120 therebetween by one or more intermolecular forces, thereby rendering the outer base layer 116 and the inner coating layer 118 sufficiently inseparable in accordance with an intended use of the implant-delivery funnel 100, as set forth below. Further, if the inner coating layer 118 does not include an antimicrobial absorbed or otherwise integrated therein as set forth below, the funnel body 102 can include an innermost antimicrobial layer 122, any of which functions to reduce opportunities for contamination of surgical pockets for implants. Regardless of whether the inner coating layer 118 includes the antimicrobial, the innermost antimicrobial layer 122 is present, or the funnel body 102 includes any antimicrobial means for reducing or eliminating microbes within the funnel body 102, the funnel body 102 can further include an outermost antimicrobial layer 124 in some embodiments. Such an outermost antimicrobial layer 124 reduces opportunities for contamination of the surgical pockets with skin flora, thereby further reducing the risks of contamination and capsular contraction over that of the existing implant-delivery funnels set forth above. Notably, the outer face 114 of the outer base layer 116 can be functionalized as set forth below for the mating face 126 of the outer base layer 116, which can be advantageous for likewise rendering the outer base layer 116 and the outermost antimicrobial layer 124 sufficiently inseparable in accordance with the intended use of the implant-delivery funnel 100.

The outer base layer 116 can include one or more layers selected from various synthetic polymers. Indeed, the one-or-more layers of the outer base layer 116 can be selected from at least a polyvinyl chloride, a polyethylene, and a polyester. In an example, the outer base layer 116 can include one or more layers of a polyvinyl chloride. In another example, the outer base layer 116 can include at least one layer of a polyethylene and at least one layer of a polyester. Notably, the example polymers are given as genera thereof as different monomers of a same type (e.g., different esters), different polymerization conditions, different additives (e.g., stabilizers, plasticizers, fillers, extenders, etc.), and the like can lead to different species of the example polymers with different properties, of which different properties at least tensile strength is important for structural integrity of the funnel body 102. While the outer base layer 116 can include one or more plasticizers for its suppleness, the outer base layer 116 is free of any phthalate plasticizers in some embodiments.

The outer base layer 116 can include a mating face 126. If the outer base layer 116 includes a plurality of layers selected from the various synthetic polymers set forth above, an innermost layer of the plurality of layers includes the mating face 126. Again, the outer base layer 116 and the inner coating layer 118 can be anchored together at the interface 120 therebetween by the one-or-more intermolecular forces, which, in more detail, means that at least the mating face 126 of the outer base layer 116 and the mating face 128 of the inner coating layer 118 are anchored together at the interface 120 therebetween by the one-or-more intermolecular forces, thereby rendering the outer base layer 116 and the inner coating layer 118 sufficiently inseparable in accordance with the intended use of the implant-delivery funnel 100. Notably, inseparability of the outer base layer 116 and the inner coating layer 118 reduces or prevents binding or filling of the surface pores of at least the polyurethane-coated silicone implants set forth above, thereby preserving the opportunities for the tissue adherence or ingrowth into the polyurethane and limiting the opportunities for malposition of the polyurethane-coated silicone in their surgical pockets.

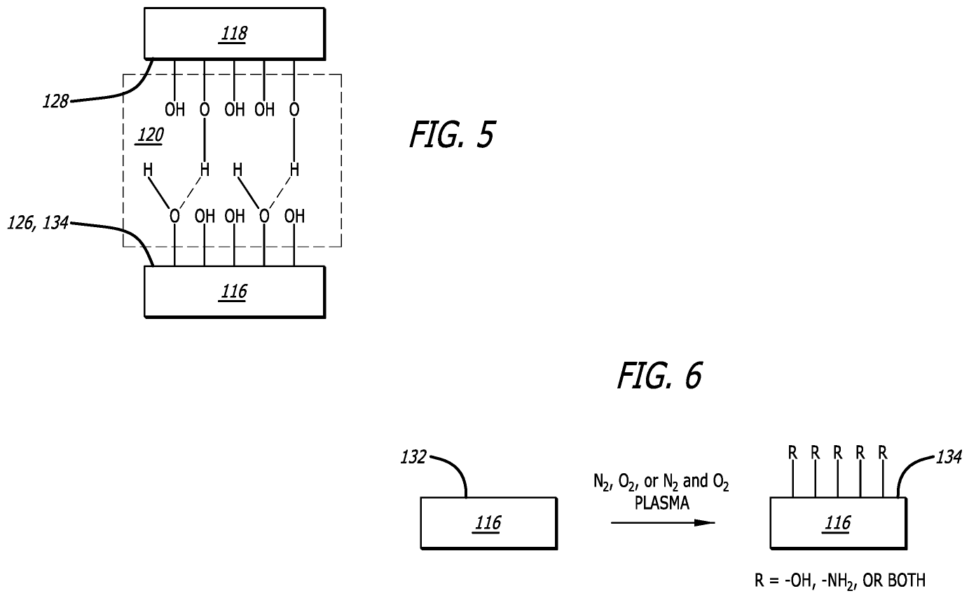

FIG. 5 illustrates the one-or-more intermolecular forces between the mating face 126 of the outer base layer 116 and the mating face 128 of the inner coating layer 118 together in accordance with some embodiments.

The one-or-more intermolecular forces are selected from ion-dipole forces, ion-induced dipole forces, dipole-dipole forces (e.g., hydrogen bonding), dipole-induced dipole forces, and London dispersion forces. Each face of the mating face 126 of the outer base layer 116 and the mating face 128 of the inner coating layer 118 can be appropriately modified, as needed, for the anchoring of the mating face 126 of the outer base layer 116 and the mating face 128 of the inner coating layer 118 together by the one-or-more intermolecular forces. In an example, the one-or-more intermolecular forces by which the outer base layer 116 and the inner coating layer 118 are anchored together can be hydrogen bonding, as shown by example with hydroxyl functional groups, which hydrogen bonding is understood to be a relatively strong dipole-dipole force. Should the outer base layer 116 be, for example, any of the polyvinyl chloride, the polyethylene, or the polyester set forth above, the mating face 126 of the outer base layer 116 can be functionalized as shown in FIG. 6 with amino functional groups, hydroxyl functional groups, or both the amino and hydroxyl functional groups as a result of the plasma functionalization set forth below for the hydrogen bonding with the mating face 128 of the inner coating layer 118 at the interface 120. Notably, if the mating face 128 of the inner coating layer 118 includes any of the various biopolymers set forth below, the mating face 128 of the inner coating layer 118 need not be further functionalized for the hydrogen bonding as the mating face 128 of the inner coating layer 118 already includes functional groups capable of hydrogen bonding such as amino functional groups, hydroxyl functional groups, carboxylic-acid functional groups, or derivatives thereof such as amides.

The inner coating layer 118 can include one or more layers selected from various biopolymers, optionally, those that are not only biocompatible but bioresorbable for naturally degrading in a human body without inflammation should any portion of the inner coating layer 118 inadvertently separate from the outer base layer 116. Indeed, the one-or-more layers of the inner coating layer 118 can be selected from natural biopolymers, semisynthetic biopolymers, or synthetic biopolymers, any of which can further include cross-links, thereby being cross-linked biopolymers of natural, semisynthetic, or synthetic origin. However, the one-or-more layers of the inner coating layer 118 need not include such cross-links. In an example, any layer or layers of the one-or-more layers of the inner coating layer 118 can independently include a natural, semisynthetic, or synthetic cross-linked biopolymer, the cross-linked biopolymer forming a three-dimensional polymer network in accordance with its cross-links. Such a cross-linked biopolymer can be solvophilic with the polymer network thereof configured to swell in a solvent or a solvent mixture by absorbing at least the solvent into the polymer network, thereby conferring additional physical properties to the cross-linked biopolymer. Specifically, such a cross-linked biopolymer can be hydrophilic with the polymer network thereof configured to hydrate and swell in water or an aqueous mixture such as an aqueous solution (e.g., physiological saline, aqueous chlorhexidine, Dakin's solution, etc.) or an aqueous suspension by absorbing at least the water into the polymer network, thereby conferring additional physical properties to the cross-linked biopolymer such as lubricity for a lubricious coating with retained water. Further, when the foregoing cross-linked biopolymer is treated with the aqueous mixture such as the aqueous solution or the aqueous suspension, the polymer network thereof can be further configured to absorb any solute (e.g., sodium chloride, any of various chlorhexidine salts, sodium hypochlorite, etc.) of the aqueous solution or suspended particles of the aqueous suspension into the polymer network, thereby conferring therapeutic properties to the cross-linked biopolymer such as antimicrobial activity.

Notably, the dynamic coefficient of friction of the inner coating layer 118 can be smaller when it is hydrated than when it is not hydrated (i.e., dry), thereby providing sliding lubriciousness to the inner coating layer 118 or the lubricious coating thereof. Such a smaller dynamic coefficient of friction results in smaller forces acting against movement of the implant 110 within the funnel body 102 making it easier to slide the implant 110 therethrough.

Like the outer base layer 116, the inner coating layer 118 can include a mating face 128, which, as set forth above, can be anchored to the mating face 126 of the outer base layer 116 at the interface 120 between the outer base layer 116 and the inner coating layer 118. Further, the inner coating layer 118 can include an exposed, inner face 130, particularly in embodiments of the implant-delivery funnel 100 not including the innermost antimicrobial layer 122. In embodiments of the implant-delivery funnel 100 including the innermost antimicrobial layer 122, the inner face 130 of the inner coating layer 118 can instead be referred to as an opposite mating face of the inner coating layer 118; however, the opposite mating face of the inner coating layer 118 need not be anchored to a mating face of the innermost antimicrobial layer 122. Regardless, if the inner coating layer 118 includes a plurality of layers selected from the various biopolymers, an innermost layer of the plurality of layers includes the inner face 130 or the opposite mating face. And while any layer or layers of the one-or-more layers of the inner coating layer 118 can independently include the cross-linked biopolymer, at least the innermost layer of the foregoing plurality of layers includes the cross-linked biopolymer in embodiments of the implant-delivery funnel 100 not including the innermost antimicrobial layer 122. Indeed, the additional physical and therapeutic properties set forth above such as lubricity and antimicrobial activity can be conferred to the cross-linked biopolymer, so it is preferable the innermost layer of the foregoing plurality of layers having the inner face 130 include the additional physical and therapeutic properties.

### Methods

Methods include methods of making the implant-delivery funnel 100 and methods of using the implant-delivery funnel 100.

A method of making the implant-delivery funnel 100 can include one or more steps selected from a base layer-forming operation, a functionalizing operation, a coating layer-applying operation, a drying operation, an indicia-creating operation, a welding operation, a hydrating operation, an antimicrobial-applying operation, and a sterilizing operation.

The base layer-forming operation can include forming the outer base layer 116 of the funnel body 102 in a flattened form thereof. Such a base-layer forming operation can include film casting, injection molding, blow molding, compression molding, extrusion, thermoforming, or the like, optionally, paired with any cutting, trimming, or the like to form the outer base layer 116 of the funnel body 102.

FIG. 6 illustrates the functionalizing operation in accordance with some embodiments.

As shown, the functionalizing operation can include functionalizing a non-functionalized mating face 132 of the outer base layer 116. Such a functionalizing operation can include functionalizing the non-functionalized mating face 132 of the outer base layer 116 with the amino functional groups, the hydroxyl functional groups, or both the amino and hydroxyl functional groups set forth above via plasma functionalization to form the outer base layer 116 with a functionalized mating face 134. (Notably, the functionalized mating face 134 can be identical to the mating face 126 of the outer base layer 116 set forth above having the amino or hydroxyl functional group.) In an example, the non-functionalized mating face 132 of the outer base layer 116 can be functionalized with the amino functional groups via plasma functionalization in an atmosphere of nitrogen and, therefore, nitrogen plasma. In another example, the non-functionalized mating face 132 of the outer base layer 116 can be functionalized with the hydroxyl functional groups via plasma functionalization in an atmosphere of oxygen and, therefore, oxygen plasma. In another example, the non-functionalized mating face 132 of the outer base layer 116 can be functionalized with the amino and hydroxyl functional groups via plasma functionalization in an atmosphere of natural or synthetic air and, therefore, at least nitrogen and oxygen plasma.

The coating layer-applying operation can include applying the inner coating layer 118 over the functionalized mating face 134 of the outer base layer 116, thereby anchoring the inner coating layer 118 to the outer base layer 116 by the dipole-dipole forces set forth above, which include the hydrogen bonding shown in FIG. 5. Notably, the applying of the inner coating layer 118 over the functionalized mating face 134 of the outer base layer 116 can include dip coating, brushing, roll coating, spray coating, spin coating, flow coating, or the like.

The drying operation can include drying the outer base layer 116 with the inner coating layer 118 anchored thereto.

The indicia-creating operation can include creating the indicia 112 on the outer face 114 of the outer base layer 116 in the distal portion thereof. Such an indicia-creating operation can include printing lines on the outer face 114 of the outer base layer 116, optionally, with corresponding labels according to implant size. Notably, the lines printed on the outer face 114 of the outer base layer 116 can be linear lines, the indicia 112 becoming the annular lines set forth above subsequent to the welding operation, which requires rolling the outer base layer 116 into the cone shape of the funnel body 102.

Notably, as in order with that set forth above, the creating of the indicia 112 on the outer face 114 of the outer base layer 116 in the indicia-creating operation can be after the applying of the inner coating layer 118 over the functionalized mating face 134 of the outer base layer 116 in the coating layer-applying operation. However, the creating of the indicia 112 on the outer face 114 of the outer base layer 116 in the indicia-creating operation can be before the functionalizing of the non-functionalized mating face 132 of the outer base layer 116 in the functionalizing operation, which functionalizing operation should be before the applying of the inner coating layer 118 in the coating layer-applying operation, particularly, if the functionalizing of the non-functionalized mating face 132 of the outer base layer 116 is needed to anchor the outer base layer 116 and the inner coating layer 118 together at the interface 120 therebetween by the one-or-more intermolecular forces. Lastly, the creating of the indicia 112 on the outer face 114 of the outer base layer 116 in the indicia-creating operation can be after the functionalizing of the non-functionalized mating face 132 of the outer base layer 116 in the functionalizing operation in order with that set forth above but before the applying of the inner coating layer 118 over the functionalized mating face 134 of the outer base layer 116 in the coating layer-applying operation.

The welding operation can include welding together longitudinal ends of the funnel body 102 present in the flattened form thereof to form the funnel opening 104 at the proximal end of the funnel body 102 and the funnel exit 106 at the distal end of the funnel body 102. Such a welding operation can include welding together the funnel body 102 by radiofrequency welding; however, the welding operation is not limited thereto as laser welding, solvent welding, or any of a number of welding techniques can be used in the welding operation.

Notably, as in order with that set forth above, the welding together of the funnel body 102 in the welding operation can be after both the functionalizing of the non-functionalized mating face 132 of the outer base layer 116 in the functionalizing operation and the applying of the inner coating layer 118 over the functionalized mating face 134 of the outer base layer 116 in the coating layer-applying operation. However, the welding together of the funnel body 102 in the welding operation can be before the functionalizing of the non-functionalized mating face 132 of the outer base layer 116 in the functionalizing operation, which functionalizing operation should be before the applying of the inner coating layer 118 in the coating layer-applying operation, particularly, if the functionalizing of the non-functionalized mating face 132 of the outer base layer 116 is needed to anchor the outer base layer 116 and the inner coating layer 118 together at the interface 120 therebetween by the one-or-more intermolecular forces. Lastly, the welding together of the funnel body 102 in the welding operation can be after the functionalizing of the non-functionalized mating face 132 of the outer base layer 116 in the functionalizing operation in order with that set forth above but before the applying of the inner coating layer 118 over the functionalized mating face 134 of the outer base layer 116 in the coating layer-applying operation.

The hydrating operation can include hydrating the inner coating layer 118 with the water or the aqueous mixture set forth above to form the lubricious coating with the retained water. As set forth above, the inner coating layer 118 can include the one-or-more layers of the cross-linked biopolymer, which forms the polymer network configured to swell with the retained water. However, it should be understood that the hydrating operation need not be performed if the implant-delivery funnel 100 is not to be pre-lubricated. Indeed, the implant-delivery funnel 100 can be easier to sterilize and package when not pre-lubricated. Further, the implant-delivery funnel 100 can have a longer shelf life when not pre-lubricated.

The antimicrobial-applying operation can include applying the innermost antimicrobial layer 122, the outermost antimicrobial layer 124, or both the innermost antimicrobial layer 122 and the outermost antimicrobial layer 124, wherein the applying of both the innermost antimicrobial layer 122 and the outermost antimicrobial layer 124 occurs separately or together in the antimicrobial-applying operation.

Notably, the hydrating operation of hydrating the inner coating layer 118 with the aqueous mixture set forth above can obviate the applying of the innermost antimicrobial layer 122 in the antimicrobial-applying operation. Indeed, the hydrating operation can include hydrating the inner coating layer 118 with the aqueous mixture set forth above such as the aqueous solution or the aqueous suspension, which can include solutes or suspended particles having therapeutic properties such as antimicrobial activity. And since the one-or-more layers of the inner coating layer 118 can include the cross-linked biopolymer having the polymer network configured to absorb such solutes or suspended particles along with water, the inner coating layer 118 can provide sufficient antimicrobial activity within the funnel body 102 for reducing or eliminating microbes therein.

The sterilizing operation can include sterilizing the implant-delivery funnel 100 before or after any one or more operations of the method set forth above for making the implant-delivery funnel 100. Additionally or alternatively, the sterilizing operation can include sterilizing the implant-delivery funnel 100 during or after packaging the implant-delivery funnel 100. Such sterilizing can include sterilizing the implant-delivery funnel 100 or a nascent form thereof, optionally, in some manner of packaging, by way of dry heat, steam, vaporized hydrogen peroxide, vaporized peracetic acid, ethylene oxide, chlorine dioxide, nitrogen dioxide, gamma radiation, electron-beam radiation, or the like.

A method of using the implant-delivery funnel 100 can include one or more steps selected from a funnel-sizing operation, a funnel-hydrating operation, a funnel-loading operation, and an implant-delivering operation.

FIG. 1 illustrates aspects of the funnel-sizing operation and the funnel-loading operation in accordance with some embodiments.

As shown, the funnel-sizing operation can include cutting the distal portion of the funnel body 102 to size in accordance with the indicia 112 of the distal portion of the funnel body 102 such that the funnel exit 106 is appropriately sized to deliver the implant 110 (e.g., a breast implant) squeezed therethrough with the temporary reduction in the size of the implant 110. Notably, the cutting of the distal portion of the funnel body 102 to size can include opening the distal portion of the funnel body 102 by excising any transverse seam in the distal portion of the funnel body 102, thereby forming the funnel exit 106 at the distal end of the funnel body 102.

The funnel-hydrating operation can include hydrating the inner coating layer 118 of the funnel body 102 with water or an aqueous mixture such as an aqueous solution or aqueous suspension to form the lubricious coating with the retained water. However, the funnel-hydrating operation need not be performed if it was already included in the method of making the implant-delivery funnel 100 set forth above. Said differently, if the implant-delivery funnel 100 is pre-lubricated during manufacturing of the implant-delivery funnel 100, the funnel-hydrating operation need not be performed in the method of using the implant-delivery funnel 100. Regardless, it can be beneficial for the inner coating layer 118 of the funnel body 102 to be hydrated to form the lubricious coating before the funnel-loading operation.

Notably, the hydrating of the inner coating layer 118 of the funnel body 102 in the funnel-hydrating operation can be performed through the funnel exit 106 at the distal end of the funnel body 102. However, if a transverse seam exists in the distal portion of the funnel body 102, the funnel-sizing operation should be performed to create the funnel exit 106 at the distal end of the funnel body 102 before the funnel-hydrating operation is performed therethrough. Alternatively, the hydrating of the inner coating layer 118 of the funnel body 102 in the funnel-hydrating operation can be performed through the funnel opening 104 at the proximal end of the funnel body 102. However, if any transverse seam exists in the proximal portion of the funnel body 102, at least a slit should be made in the proximal portion of the funnel body 102 to create a corresponding opening before the funnel-hydrating operation is performed therethrough. Advantageously, when the slit or the corresponding opening in the proximal portion of the funnel body 102 is sufficiently large to accommodate the implant 110, the funnel-loading operation can be performed therethrough without excising an entirety of the transverse seam in the proximal portion of the funnel body 102 to create the funnel opening 104.

The funnel-loading operation can include dropping or inserting the implant 110 into the funnel opening 104 as further shown in FIG. 1. Again, the funnel opening 104 can be sized larger than the implant 110 such that the implant 110 need not be manipulated when dropped or inserted into the funnel opening 104, which allows minimal to no direct handling of the implant 110, thereby reducing any potential for contamination. Regardless of any direct handling of the implant 110 during the funnel-loading operation, the implant 110 need not be further handled subsequent to the funnel-loading operation.

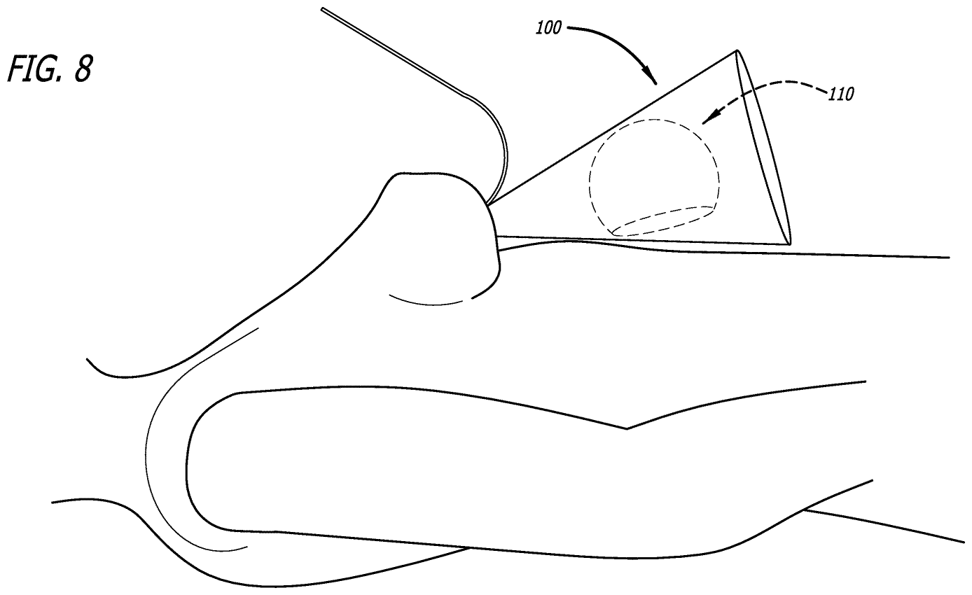

FIG. 7 illustrates squeezing the funnel body 102 proximal of the implant 110 disposed therein to deliver the implant 110 in accordance with some embodiments. FIG. 8 illustrates implantation of the implant 110 in a patient via delivery of the implant 110 with the implant-delivery funnel 100 in accordance with some embodiments.

As shown, the implant-delivering operation can include inserting the distal portion of the funnel body 102 through an incision sized smaller than the implant 110 and into a surgical pocket sized to accept the implant 110 at its full size. Further, the implant-delivering operation can include squeezing the funnel body 102 proximal of the implant 110 disposed therein and, while maintaining applied pressure by the squeezing, working both hands distally over the funnel body 102 until the implant 110 is just proximal of the funnel exit 106 or protrudes from the funnel exit 106. Additional squeezing of the funnel body 102 proximal of the implant 110 disposed therein squeezes the implant 110 through the funnel exit 106 with the temporary reduction in the size of the implant 110, so as to pass the implant 110 through the smaller-sized incision, and into the surgical pocket where the implant 110 assumes its full size.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. An implant-delivery funnel, comprising:
a supple, layered funnel body including:
an outer base layer; and
an inner coating layer anchored to the outer base layer by dipole-dipole forces, the inner coating layer being hydrophilic such that the inner coating layer forms a lubricious coating with retained water;
a funnel opening at a proximal end of the funnel body, the funnel opening sized to receive an implant therethrough without any permanent deformation of the implant; and
a funnel exit at a distal end of the funnel body, the funnel exit adjustably sized to deliver the implant squeezed therethrough with a temporary reduction in size of the implant.

2. The implant-delivery funnel according to claim 1, wherein the outer base layer includes one or more layers of polyvinyl chloride.

3. The implant-delivery funnel according to claim 1, wherein the outer base layer includes at least one layer of polyethylene and at least one layer of polyester.

4. The implant-delivery funnel according to claim 1, wherein the outer base layer is free of any phthalate plasticizers.

5. The implant-delivery funnel according to claim 1, wherein the dipole-dipole forces include hydrogen bonding.

6. The implant-delivery funnel according to claim 5, wherein a mating face of the outer base layer is functionalized with amino functional groups, the inner coating layer anchored to the outer base layer by the hydrogen bonding.

7. The implant-delivery funnel according to claim 5, wherein a mating face of the outer base layer is functionalized with amino functional groups, the inner coating layer anchored to the outer base layer by the hydrogen bonding.

8. The implant-delivery funnel according to claim 5, wherein a mating face of the outer base layer is functionalized with amino and hydroxyl functional groups, the inner coating layer anchored to the outer base layer by the hydrogen bonding.

9. The implant-delivery funnel according to claim 1, wherein the inner coating layer is hydrated with water or an aqueous mixture, thereby forming the lubricious coating with the retained water.

10. The implant-delivery funnel according to claim 9, wherein the inner coating layer includes one or more layers of a cross-linked biopolymer forming a polymer network configured to swell with the retained water.

11. The implant-delivery funnel according to claim 10, wherein the inner coating layer is further antimicrobial, the polymer network configured to swell with the retained water and any antimicrobial dissolved or suspended in the aqueous mixture.

12. The implant-delivery funnel according to claim 1, the funnel body further including an innermost antimicrobial layer.

13. The implant-delivery funnel according to claim 1, the funnel body further including an outermost antimicrobial layer.

14. The implant-delivery funnel according to claim 1, wherein the funnel body includes a longitudinal welded seam.

15. The implant-delivery funnel according to claim 1, wherein the funnel body includes a transverse welded seam.

16. The implant-delivery funnel according to claim 1, wherein the implant-delivery funnel is configured to deliver breast implants of various volumes and shapes.

17. The implant-delivery funnel according to claim 1, wherein a distal portion of the funnel body includes indicia configured for cutting the distal portion of the funnel body to size such that the funnel exit is sized to deliver the implant squeezed therethrough with the temporary reduction in size of the implant.

18. A method of making an implant-delivery funnel, comprising:
forming an outer base layer of a funnel body;
functionalizing a mating face of the outer base layer;
applying an inner coating layer over a functionalized mating face of the outer base layer, thereby anchoring the inner coating layer to the outer base layer by dipole-dipole forces including hydrogen bonding; and
welding together the funnel body to form:
a funnel opening at a proximal end of the funnel body, the funnel opening sized to receive an implant therethrough without any permanent deformation of the implant; and
a funnel exit at a distal end of the funnel body, the funnel exit adjustably sized to deliver the implant squeezed therethrough with a temporary reduction in size of the implant.

19. The method according to claim 18, wherein the welding together of the funnel body is before the functionalizing of the mating face of the outer layer.

20. The method according to claim 18, wherein the welding together of the funnel body is after the functionalizing of the mating face of the outer layer but before the applying of the inner coating layer over the functionalized mating face of the outer base layer.

21. The method according to claim 18, wherein the welding together of the funnel body is after both the functionalizing of the mating face of the outer layer and the applying of the inner coating layer over the functionalized mating face of the outer base layer.

22. The method according to claim 18, wherein the welding together of the funnel body is by radiofrequency welding.

23. The method according to claim 18, wherein the functionalized mating face of the outer base layer is functionalized with amino functional groups via plasma functionalization in an atmosphere of nitrogen, the inner coating layer anchored to the outer base layer by the hydrogen bonding.

24. The method according to claim 18, wherein the functionalized mating face of the outer base layer is functionalized with amino and hydroxyl functional groups via plasma functionalization in an atmosphere of natural or synthetic air, the inner coating layer anchored to the outer base layer by the hydrogen bonding.

25. The method according to claim 18, further comprising hydrating the inner coating layer with water or an aqueous mixture to form a lubricious coating with retained water, the inner coating layer including one or more layers of a cross-linked biopolymer forming a polymer network configured to swell with the retained water.

26. The method according to claim 25, wherein the inner coating layer is further antimicrobial, the polymer network configured to swell with the retained water and any antimicrobial dissolved or suspended in the aqueous mixture.

27. The method according to claim 18, further comprising applying an innermost antimicrobial layer.

28. The method according to claim 18, further comprising applying an outermost antimicrobial layer.

29. The method according to claim 18, further comprising creating indicia on an outer face of the outer base layer in a distal portion thereof, the indicia configured for cutting the distal portion of the funnel body to size such that the funnel exit is sized to deliver the implant squeezed therethrough with the temporary reduction in size of the implant.
